(19) United States
(12) Patent Application Publication
(10) Pub. No.: US 2024/0361316 A1
(43) Pub. Date: Oct. 31, 2024

(54) COVID-19 IMMUNE RESPONSE ASSAY METHOD

(71) Applicant: ORASURE TECHNOLOGIES, INC., Bethlehem, PA (US)

(72) Inventors: Janean A. YOUNG, Bethlehem, PA (US); Jody BERRY, Bethlehem, PA (US)

(21) Appl. No.: 18/291,711

(22) PCT Filed: Jul. 25, 2022
(86) PCT No.: PCT/US2022/074112

§ 371 Date: Jan. 24, 2024

Related U.S. Application Data
(60) Provisional application No. 63/225,512, filed on Jul. 25, 2021.

(51) Int. Cl. G01N 33/569

(52) U.S. Cl. CPC G01N 33/56983; G01N 2333/165; G01N 2469/20

(57) ABSTRACT

Disclosed herein are assay methods and kits for assessing the strength of the immune response to SARS-CoV-2 virus in a subject by allowing for rapid and efficient detection of neutralizing antibodies (NAbs) from serum of vaccinated and non-vaccinated subjects. The assay method can be conducted in a biosafety level 2 setting, and uses highly-purified, monomeric SARS-Cov-2-Spike1 which leads to improved sensitivity and specificity over current state of the art assays.

### CROSS REFERENCE TO RELATED APPLICATIONS

This application claims priority to U.S. Provisional Application No. 63/225,512, filed on Jul. 25, 2021, the disclosure of which is incorporated by reference.

### SEQUENCE LISTING

The contents of the electronic sequence listing (2022-07-25_PCT_Sequence_Listing.xml; Size: 8,620 bytes; and Date of Creation: Jul. 25, 2022) is herein incorporated by reference in its entirety.

### TECHNICAL FIELD

This disclosure relates to method for the detection of neutralizing antibodies against SARS-CoV-2 Spike protein, termed iNab (in vitro neutralizing antibody assay).

### BACKGROUND

COVID-19 is the disease caused by SARS-CoV-2 coronavirus. COVID-19 spreads when an infected person breathes out droplets and particles containing the virus. Those droplets and particles may then be breathed in by other people or may land on their eyes, noses and/or mouth and cause infection. COVID-19 can be spread by infected but asymptomatic persons. Before the development of vaccines, wearing a mask and social-distancing were the primary protections against COVID-19. Worldwide, billions of COVID-19 vaccines have been administered individuals to prevent hospitalizations and deaths. However, even with vaccines available, there remains a need for assay methods and devices for assessing immune responses to SARS-CoV-2, including methods for detecting the presence of neutralizing antibodies in vaccinated and unvaccinated people.

The spike (S) glycoprotein of a coronavirus, which is also commonly referred to as spike protein, is the largest of the four major structural proteins of coronaviruses. The function of the spike glycoprotein is to mediate viral entry into the host cell by first interacting with molecules on the exterior cell surface, and then, fusing the viral and cellular membranes. Spike glycoprotein is a class I fusion protein, which is composed of two subunits, S1 and S2. The S1 subunit contains the receptor-binding domain (RBD), which functions to bind coronaviruses to receptors on the surface of host cells.

The RBD of the S1 subunit of SARS-CoV-2 spike protein binds human angiotensin-converting enzyme 2 (ACE2). Upon the binding interaction of RBD and ACE2, a conformational change occurs in SARS-CoV-2 viral proteins, which, in turn, causes viral uptake by the host cell, infection, and replication of the virus in the host cell. Antibodies that specifically bind the RBD region of Spike 1 can block the interaction with ACE2 and thereby inhibit or neutralize the infection.

Naturally infected and vaccinated people may be capable of producing antibodies that will block the interaction of the S1 RBD with ACE2, and thus, inhibit viral entry and replication in those individuals. However, the degree to which an individuals can mount an antibody response to SARS-CoV-2, and, more particularly, the S1 subunit, varies within a population, as well as by a person over time. Thus, there is a need for assays that can be used to evaluate the strength, or in other words, the robustness, of an antibody response to SARS-CoV-2. In that regard, assessing a person's antibody response to the S1 subunit, especially against its RBD, is critical to determine whether the antibodies are capable of neutralizing SARS-CoV-2, and, thus capable of blocking the SARS-CoV-2 infection cycle.

Current methods for assessing neutralization of SARS-CoV-2, generally known in the art as viral neutralization (VNT) requires biosafety level 3 (BSL3) laboratory conditions, including medical surveillance of personnel, protective gear, and laboratory building, air flow, and containment requirements. These requirements are onerous and expensive, and, ultimately, slow determinations of individual and herd immunity to SARS-CoV-2. The development of a simple, quick, BSL2, plate-based method for detecting antibodies capable of competing with S1 subunits for binding to ACE2, will shorten the time and expense for determining the presence of neutralizing antibodies (NAbs) in the serum of vaccinated and non-vaccinated donors. Additionally, presenting RBD in the context of highly-purified, monomeric S1 allows for improved assay sensitivity and specificity over current state of the art assays for detecting SARS-CoV-2 neutralizing antibodies.

### SUMMARY OF THE INVENTION

The invention relates to assay methods for assessing the strength of the immune response to SARS-CoV-2 virus in a subject, which has been exposed, or potentially exposed, to SARS-CoV-2 virus. Such methods of the invention include: a) contacting a solid substrate, on which a plurality of SARS-CoV-2-Spike 1 subunit proteins (S1 antigens) are bound to the surface of the substrate, with a biological sample from the subject, under reaction conditions to allow S1 antigen-specific antibodies, if present in the biological sample, to bind the S1 antigen, bound to the substrate, to form S1 antigen-antibody complexes on the surface of the substrate; b) contacting the S1 antigen-antibody complexes on the surface of the substrate with a solution comprising a plurality of biotinylated angiotensin-converting enzyme 2 (ACE2) proteins under conditions to allow the biotinylated ACE2 proteins to compete for binding to the S1 antigen with the complexed S1 antigen-specific antibodies in (a); c) contacting the solution of (b) with a solution comprising streptavidin labeled with a detection moiety, under conditions to allow labelled streptavidin-biotin complexes to form; and d) detecting the labelled streptavidin-biotin complexes, wherein the strength of the immune response to S1 antigen corresponds to the level of label detected, wherein the level of detected label corresponds to inhibition of ACE2 protein binding to the S1 antigen by the S1 antigen-specific antibodies in the biological sample.

Usually, a biological sample that is obtained from a patient for analysis by a method of the invention is blood or the serum fraction of blood. However, in some methods of the invention, the biological sample may be saliva, sputum or other liquids or solid substances obtained from a patient. Irrespective of the source of a biological sample of the invention, the sample may be heat inactivated prior to being used in a method of the invention. The sample is also typically diluted for use in methods of the invention, usually from 1:10 to 1:20,000 (Sample:Diluent).

With respect to the S1 subunit used methods of the invention, the plurality of SARS-CoV-2-Spike 1 subunit proteins (S1 antigens) are typically S1 subunit monomers. The S1 subunit used in a method of the invention may be based on the S1 subunit protein of any variant of SARS-CoV-2, including, but not limited to the S1 subunit of any one of the following SARS-CoV-2 variants: WHO Alpha (WT); WHO Delta; U.K. (alpha, B.1.1.7); South African (beta, B.1.351); Brazil (gamma, P.1); India (delta, B.1.617.2); California (epsilon, B.1.429/427); and Epsilon; Epsilon BA.1; Epsilon BA.2; Epsilon BA.3; Epsilon BA.4; and Epsilon BA.5. In certain methods of the invention, the plurality of S1 subunit proteins used in the assay are based on the amino sequence of any one of SEQ ID NOs. 3-5, including S1 subunit proteins that are at least 80%, at least 85%, at least 90%, at least 95%, at least 96%, at least 97%, at least 98%, or at least 99% to the amino acid sequence of any one of SEQ ID Nos: 3-5. Furthermore, in methods of the invention, the S1 subunit protein may include the receptor-binding domain (RBD) of the S1 subunit.

With respect to the solid substrate used in a method of the invention, the solid substrate may be, but is not necessarily required to be, a glass or a polymer material. For example, a polymer may be, but is not limited to, cellulose, polyacrylamide, nylon, polystyrene, polyvinylchloride, or polypropylene. The form of the solid substrate used in a method of the invention may be in any form appropriate for performing the reaction steps of the method, For example, in some methods of the invention, the solid substrate may be the surface of a tube or similar reaction vessel, like a microtube coated with a plurality of S1 subunit proteins; a protein that is coated or otherwise complexed with a plurality of S1 subunit proteins; a bead coated with a plurality of S1 subunit proteins, a disc coated with a plurality of S1 subunit proteins, or the wells of a microplate that have been coated with a plurality of S1 subunit proteins.

With respect to the angiotensin-converting enzyme 2 (ACE2) used in some methods of the invention, the ACE2 protein contains an amino acid sequence of any one of SEQ ID NOs. 1 or 2. For example, in some methods of the invention, the ACE2 protein is at least 80%, at least 85%, at least 90%, at least 95%, at least 96%, at least 97%, at least 98%, or at least 99% to the amino acid sequence of any one of SEQ ID Nos: 1 or 2. In the same or different methods of the invention, the ACE2 proteins may be a fusion protein of ACE2 and an N-terminal affinity tag or a C-terminal affinity tag. For example, the N-terminal affinity tag or the C-terminal affinity tag of an ACE2 protein of the invention may be a poly-histidine tag or a histidine-arginine tag.

In some methods of the invention, if the strength of the immune response to SARS-CoV-2 virus in a subject is low, the subject is administered treatment for SARS-CoV-2. In other methods of the invention, the subject is need of an organ transplant, and if the strength of the immune response to SARS-CoV-2 virus in the subject is high, then the subject receives the organ transplant, whereas if the strength of the immune response to SARS-CoV-2 virus in a subject is low, then the subject does not receive the organ transplant.

In some methods of the invention, the assay method further includes performing the method with a negative control and a positive control, wherein the negative control does not include biotinylated ACE2 proteins, and the positive control is performed using known neutralizing antibodies that are specific for the RBD of the SARS-CoV-2 S1 subunit. In such methods on the invention, the percent inhibition of antibodies in the biological sample may be calculated using the following formula:

$$\% Inhibition = ( 1 - \frac{S - {C}_{min}}{{C}_{max} - {C}_{min}} ) \times 100 ;$$

wherein S is the signal detected using the biological sample, and Cₘᵢₙ and Cₘₐₓ are the negative and positive controls, respectively.

In some methods of the invention, if the strength of the immune response to SARS-CoV-2 virus in a subject is low, the subject is administered treatment for SARS-CoV-2, wherein a low strength immune response to SARS-CoV-2 virus is 25% or less inhibition.

Similarly, in other methods of the invention, wherein the subject is need of an organ transplant, if the strength of the immune response to SARS-CoV-2 virus in the subject is high, then the subject receives the organ transplant, or, alternatively, if the strength of the immune response to SARS-CoV-2 virus in a subject is low, then the subject does not receive the organ transplant, wherein a high strength immune response to SARS-CoV-2 virus is 75% or more, 80% or more, 85% or more, 90% or more, 95% or more, or 100% inhibition, and a low strength immune response to SARS-CoV-2 virus is 25% or less inhibition.

### BRIEF DESCRIPTION OF THE DRAWINGS

FIG. 1 is a schematic of the in vitro neutralizing antibody assay (iNab) compared to Genscript's cPASS™ neutralizing antibody detection assay. The unique format for iNab's functional output is shown with recombinant SARS-CoV-2 Spike1 (S1) monomer coated on a 96-well plate; in the absence of anti-S1 neutralizing serum antibodies, recombinant biotinylated human angiotensin-converting enzyme 2 (ACE2;ACE2-biotin) can bind to the RBD receptor presented in properly folded S1 monomer, followed by detection using streptavidin-HRP to produce a colorimetric signal at OD 450 nm; conversely, in the presence of anti-S1 neutralizing antibodies, ACE2-biotin is blocked from binding RBD receptor within S1 coated on the plate, thus no HRP colorimetric signal develops.

FIG. 2A is a chromatograph of purified recombinant ACE2 protein characterized by SEC-HPLC before polish chromatography.

FIG. 2B is a chromatograph of purified recombinant ACE2 protein monomer characterized by SEC-HPLC after polish chromatography.

FIG. 3 depicts SDS-PAGE characterization of purified recombinant ACE2 monomer. Expected size of the recombinant ACE2 protein of ˜70 kDa correlates with observed size.

FIG. 4 depicts SDS-PAGE characterization purified recombinant SARS-CoV-2 Spike1 monomer.

FIG. 5 depicts EC₅₀ curve of iNab detection of positive control neutralizing antibody.

FIG. 6 depicts binding of ACE2-biotin to full-length recombinant S1 of iNab compared to commercially available SARS-CoV-2 RBD.

FIG. 7 depicts percent inhibition results of serum samples analyzed by iNab; dotted line represents 25% cut-off. Confirmed positive sera samples are shown in solid circles; confirmed negative sera samples are shown as hollow circles.

FIG. 8A depicts dot plot of serum samples at dilution [1:10] analyzed for present inhibition via iNab using S1 Alpha (WT) or S1 Delta coated plates; dotted line represents 25% cut-off.

FIG. 8B depicts a Before-and-After Graph representation of FIG. 9A comparing iNab results using S1 Alpha (WT) versus S1 Delta coated plates.

FIG. 9 depicts a dot plot representation of iNab results on serum samples from donors vaccinated with the Moderna COVID-19 Vaccine.

FIG. 10 depicts a dot plot representation of iNab results on serum samples from donors vaccinated with the Johnson & Johnson COVID-19 Vaccine.

FIG. 11 depicts a dot plot representation of iNab results on serum samples from COVID19-recovered donors vaccinated with the Pfizer COVID-19 Vaccine.

FIG. 12 depicts a dot plot representation of iNab results on serum samples from donors vaccinated with the Pfizer COVID-19 Vaccine.

FIG. 13 depicts the receiver operating characteristic (ROC) curve representing COVID19-vaccinated serum samples analyzed via iNab assay correlated to the gold-standard Vero E6 Virus Neutralization Test (VNT) assay.

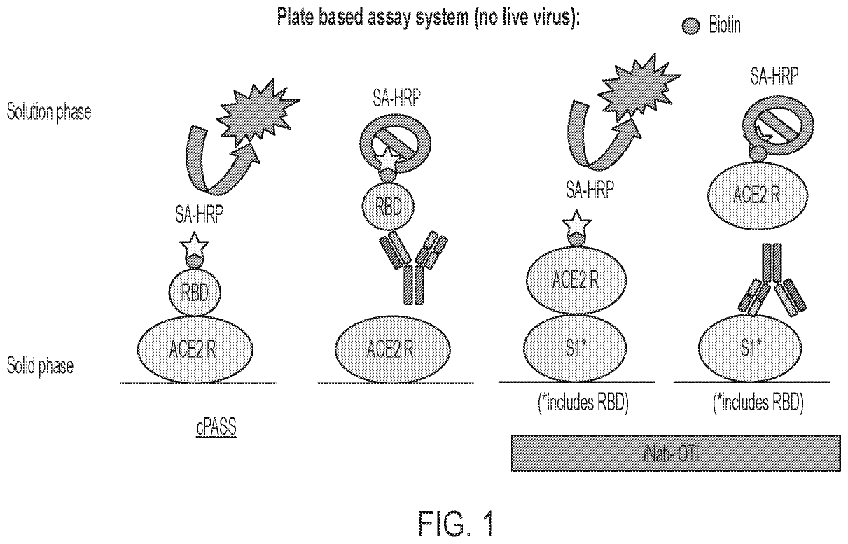

### DETAILED DESCRIPTION OF THE INVENTION

The invention relates to a quick, biosafety level 2 (BSL2), substrate-based method and device for detecting the presence or absence of neutralizing antibodies (NAbs) from serum of vaccinated and non-vaccinated donors NAbs. In a method and device of the invention, receptor binding domain (RBD) is not used as an isolate, but rather, is present within the folded structure of SARS-CoV-2 Spike1 (S1). The S1 acts as the ligand and is coated on a substrate such as an ELISA plate or a lateral flow assay strip, and biotinylated human angiotensin-converting enzyme 2 (ACE2-biotin) is the analyte to be detected. In a method of the invention, the NAbs, if present in the biological sample being tested, blocks ACE2-biotin binding to substrate-bound S1, showing evidence of neutralizing antibodies or the opposite case of non-neutralizing activity. Presenting RBD in the context of monomeric S1 in a highly purified format allows for improved assay sensitivity and specificity over current state of the art assays for detecting SARS-CoV-2 neutralizing antibodies.

The invention relates to an assay method for assessing the strength of the immune response to SARS-CoV-2 virus in a subject, which has been exposed, or potentially exposed, to SARS-CoV-2 virus, the method comprising:
a) contacting a solid substrate, on which a plurality of SARS-CoV-2-Spike 1 subunit proteins (S1 antigens) are bound to the surface of the substrate, with a biological sample from the subject, under reaction conditions to allow S1 antigen-specific antibodies, if present in the biological sample, to bind the S1 antigen, bound to the substrate, to form S1 antigen-antibody complexes on the surface of the substrate;
b) contacting the S1 antigen-antibody complexes on the surface of the substrate with a solution comprising a plurality of biotinylated angiotensin-converting enzyme 2 (ACE2) proteins under conditions to allow the biotinylated ACE2 proteins to compete for binding to the S1 antigen with the complexed S1 antigen-specific antibodies in (a);
c) contacting the solution of (b) with a solution comprising streptavidin labeled with a detection moiety, under conditions to allow labelled streptavidin-biotin complexes to form; and
d) detecting the labelled streptavidin-biotin complexes, wherein the strength of the immune response to S1 antigen corresponds to the level of label detected, wherein the level of detected label corresponds to inhibition of ACE2 protein binding to the S1 antigen by the S1 antigen-specific antibodies in the biological sample.

In one embodiment of the invention, the assay method allows for the determination of absence or presence of neutralization antibodies effective against SARS-Cov-2.

In one embodiment of the invention, the assay is an immunoassay. In a preferred embodiment, the assay is an enzyme-linked immunosorbent assay (ELISA), such as shown in the examples below.

In a method of the invention, the biological sample is blood, serum, or saliva. In a preferred method, the biological sample is serum. The biological sample may be diluted by factor of about 1:10 to about 1:50,000. For example, the biological sample may be diluted by a factor of 1:10, 1:15, 1:20, 1:25, 1:30, 1:35, 1:40, 1:45, 1:50, 1:100, 1:200, 1:500, 1:1000, 1:2000, 1:5000, 1:10000, 1:20000, or 1:50000. In a preferred embodiment of the invention, the biological sample is serum diluted by a factor of about 1:10.

In an assay method of the invention, S1 antigen is bound to a solid substrate. S1 antigen may be a purified recombinant protein construct. S1 antigen may be based on the S1 amino acid sequence of the WHO Alpha (WT) variant, the WHO Delta SARS-Cov-2 variant, or other variants. S1 antigen may comprise N- or C-terminal affinity tags, such as a poly-histidine tag or a histidine-arginine tag. In a preferred assay method, S1 antigen is monomeric. S1 antigen may have an amino sequence having at least 30%, at least 40%, at least 50%, at least 60%, at least 70%, at least 80%, or at least 90% sequence identity to any one of SEQ ID NOs. 3-5. The solid substrate can be glass or a polymer, including, but not limited to cellulose, polyacrylamide, nylon, polystyrene, polyvinylchloride, or polypropylene. The solid supports may be in the form of tubes, beads, discs, microplates, or any other surfaces suitable for conducting an immunoassay. Non-limiting examples of beads are Ni²⁺ magnetic beads, magnetic silica beads, silica beads, and cytometric beads arrays. Non-limiting examples of discs are lab-on-disc systems, e.g., polycarbonate discs or compact disc microarrays. Non-limiting examples of microplates are 96- or 394-well plates.

In an assay method of the invention, ACE2 may be a purified recombinant protein construct. An ACE2 protein construct may comprise N- or C-terminal affinity tags, such as a poly-histidine tag or a histidine-arginine tag. ACE2 may have an amino acid sequence having at least 30%, at least 40%, at least 50%, at least 60%, at least 70%, at least 80%, or at least 90% sequence identity to any one of SEQ ID NOs. 1-2. In a preferred assay method of the invention, ACE2 is monomeric.

In an assay method of the invention, ACE2 is biotinylated (ACE2-biotin). In a preferred assay method, the molar ratio of ACE2 to biotin of ACE2-biotin is about 1:1 to about 1:10.

In an assay method of the invention, the presence or absence of neutralizing antibodies is determined by measuring signal produced by labeled streptavidin binding to ACE-biotin bound to S1-coated substrate. Non-limiting examples of labels suitable for use in this aspect of the invention include radioisotopes, enzymes, or fluorophores. Commonly used enzymes include horseradish peroxidase, glucose oxidase, β-galactosidase, and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product. In a preferred assay method, the labeled streptavidin is a horse-radish peroxidase streptavidin conjugate (SA-HRP).

As used herein, the term “COVID” or “COVID-19” refers to the Coronavirus disease 2019, a contagious disease caused by severe acute respiratory syndrome coronavirus 2 (SARS-CoV-2). The term “neutralizing antibody” refers to an antibody or antibodies capable of binding to SARS-CoV-2 virus or viral protein and interfering with its ability to infect a cell. Neutralizing responses of greater than about 20-25% as determined by the iNAb assay method of the invention may indicate the presence of neutralizing antibodies in a biological sample.

In some methods of the invention, the assay method further includes performing the method with a negative control and a positive control, wherein the negative control does not include biotinylated ACE2 proteins, and the positive control is performed using known neutralizing antibodies that are specific for the RBD of the SARS-CoV-2 S1 subunit. In such methods on the invention, the percent inhibition of antibodies in the biological sample may be calculated using the following formula:

$$\% Inhibition = ( 1 - \frac{S - {C}_{min}}{{C}_{max} - {C}_{min}} ) \times 100 ;$$

and Cₘᵢₙ and Cₘₐₓ are the negative and positive controls, respectively.

In some methods of the invention, if the strength of the immune response to SARS-CoV-2 virus in a subject is low, the subject is administered treatment for SARS-CoV-2, wherein a low strength immune response to SARS-CoV-2 virus is 25% or less inhibition.

Similarly, in other methods of the invention, wherein the subject is need of an organ transplant, if the strength of the immune response to SARS-CoV-2 virus in the subject is high, then the subject receives the organ transplant, or, alternatively, if the strength of the immune response to SARS-CoV-2 virus in a subject is low, then the subject does not receive the organ transplant, wherein a high strength immune response to SARS-CoV-2 virus is 75% or more, 80% or more, 85% or more, 90% or more, 95% or more, or 100% inhibition, and a low strength immune response to SARS-CoV-2 virus is 25% or less inhibition.

In one embodiment of the invention, the assay method relates to administering a medicine for the treatment of SARS-CoV-2 in a subject in need thereof if the strength of the immune response to SARS-CoV-2 virus in the subject is low. The treatment may comprise administering to the subject an effective amound of antiviral medications and/or monoclonal or long-lasting antibody medications to treat SARS-CoV-2. Antiviral medications may target specific parts of the virus to stop it from multiplying in the body, helping to prevent severe illness and death. Non-limiting examples of antiviral medications are nirmatrelvir with ritonavir, molnupiravir, and remdesivir. Antiviral medications may be administered orally or through IV injection. Antibody medications may help the subject's immune system recognize and respond more effectively to the virus. Non-limiting examples of antibody medications are tixagevimab with cilgavimab and bebtelovimab.

In another embodiment, the invention relates to an assay method for assessing the strength of the immune response to SARS-CoV-2 virus in a subject in need of an organ transplant. If the biological sample from the subject demonstrates a strong immune response to SARS-CoV-2 virus as determined by the assay method of the invention (the iNAb assay), then the subject may possess neutralizing antibodies against SARS-Cov-2 and be able to receive an organ transplant without risk of complications from SARS-Cov-2. If the biological sample from the subject demonstrates a low immune response to SARS-CoV-2 virus as determined by the assay method of the invention (the iNAb assay), then the subject may not possess neutralizing antibodies against SARS-Cov-2 and be unable to receive an organ transplant without risk of complications from SARS-Cov-2. In this aspect, the assay method of the invention is useful for determining risk factors associated with complications from SARS-CoV-2 in subject in need of an organ transplant.

The invention also provides for assay kits. Such kits include one or more of recombinantly-produced SARS-CoV-2 Spike 1 (S1) subunit proteins and biotinylated ACE2 proteins, as well as a solution containing streptavidin labeled with a detectable moiety. In some kits of the invention, the S1 subunit proteins are monomeric, and in the same or different assay kits of the invention, the S1 subunits are attached to a solid substrate, such as, for example, the bottom of a multiwell plate (e.g., a 96 well plate used for ELISA assays). The detectable moiety component of a kit of the invention is typically a fluorescent or chemiluminescent molecule, but may also be any detectable moiety known in the art, including radioactive and enzymatic labels. A kit of the invention may also contain one or more of the buffers, reagents, detection reagents, and so forth that are useful for the practice of the methods of this invention. And certain kits of the invention may contain an assay device.

Instructional materials may also be included in a kit of the invention. The instructional materials may, in some cases, be printed on an insert included in the kits, or provided in electronic form, such as on a portable hard drive, or in a video file. Instructional materials may also refer to a website or link to an application software program, such as a mobile device or computer “App”, which provides instructions. A kit may also include additional components to facilitate the particular application for which the kit is designed. For example, a kit may also contain a means of detecting a label (such as enzyme substrates for enzymatic labels, filter sets to detect fluorescent or chemoluminescent labels, appropriate secondary labels such as a secondary antibody, or the like).

### EXAMPLES

Donor serum was evaluated with the gold standard of virus neutralization test (VNT) using live virus. Correlation between VNT and the in vitro neutralization antibody (iNab) assay (the assay method of the invention) was performed. Using the iNab assay method, the results proved robust. Comparing the samples with neutralization activity from the iNab assay to the results of live virus (VNT) assay was 100% Sensitivity and 100% Specificity using a ROC curve, P-value <0.0001. 49 donors were tested in comparison with the VNT assay. 33/33 were in 100% agreement of having neutralizing antibodies, 16/16 were in 100% agreement of not having neutralizing antibodies, of these negative samples 14/16 donors were COVID-19 negative and did not yet receive the vaccine, 2/16 were patients that received vaccines. Additional screening on the 2/16 negatives also showed no presence of anti-Spike antibodies, thus correlating with the lack of anti-Spike neutralizing antibodies. Currently CPASS assay from Genscript has a cut-off of 30% to be interpreted as have neutralizing activity using diluted serum. The iNab assay method, was determined to have a higher specificity resulting in levels down to 22-25% as positive for neutralization determination because of the use of full-length SARS-CoV-2-Spike1 (S1).

### Example 1: Human Angiotensin Converting Enzyme 2 (ACE2) Protein Constructs

The iNab surrogate neutralization assay was evaluated using (2) different designs of human ACE2. Both designs were cloned into the pcDNA™ 3.1 (+) IP free expression vector for mammalian expression, which contains an ampicillin (AMP) resistance gene, a simian virus 40 (SV40) enhancer and promoter, and a Cytomegalovirus (CMV) enhancer-promoter. Expression was performed using an Invitrogen® Expi-293 expression system in serum free media. Total expression yields were similar between the two ACE2 designs, which averaged 150 mg/L-165 mg/L of unpurified recombinant ACE2. The total expression time of 5-7 days before harvest was similar between the two ACE2 designs. Final purity, solubility, biotinylation efficiency and performance in the iNab assay were also similar between the two constructs.

Productions of each design were purified using the same method and conditions. Construct 1 (hACE-GS-8×HN; SEQ ID NO. 1) had 5-fold higher binding to the column compared to construct 2 (hACE-GS-6×HIS; SEQ ID NO. 2), thus increasing yield from capture step that would be acceptable in a manufacturing setting. Yields of purified ACE2, post nickel Sepharose® capture, were improved by using construct #1, with a shorter linker and extended Histidine-Arginine affinity tag.

In both constructs the canonical signal sequence was removed and replaced with a unique IL-2 signal sequence, thereby resulting in the starting amino acid being at 19 in isoform 1 (accession #NP_01358344) and isoform 2 (accession #NP_01373188). Each construct included different sized glycine-serine linkers for evaluating binding on nickel Sepharose® columns. Each construct added different poly-histidine based affinity tags for evaluating binding on nickel Sepharose® columns.

Human ACE2 isoforms 1 and 2 were each shortened from amino acid 708 to 591 in the processed form of ACE2. This reduction in length allowed the modified ACE2 isoforms to include the spike glycoprotein binding domain at amino acids 353-357 and the third disulfide bond region at amino acid 530 and 542 for proper structural folding. Corresponding nucleotide sequences were optimized for expression by HEK293 cells, including changes to the nucleotide sequences designed to enhance post translation modifications of the isoforms to include enhance signal peptide cleavage, glycosylation, and disulfide bond formation. Unless otherwise specified, data below were generated using the hACE-GS-6×HIS construct corresponding to SEQ ID NO. 2.

### Example 2: SARS-CoV-2 Spike 1 Subunit (S1) Protein Constructs

The iNab surrogate neutralization assay was evaluated using two different designs of SARS-CoV-2 S1 based on the WHO Alpha variant (accession #YP_009724390) and the WHO Delta variant (accession #YP_009724390), respectively. All designs were cloned into pD607, an Electra™ daughter IP-free vector system for mammalian expression, which contains a CMV/SV40 enhancer and promoter and an AMP resistance gene. Expressions were performed using an Invitrogen Expi293® expression system in serum free media. Total expression times before harvest were similar between constructs at 7-9 days. Both designs had similar binding to nickel resin during purification.

The Alpha S1 constructs (SEQ ID NOs: 3-4) and the Delta construct (SEQ ID NO. 5) were truncated to amino acid 674 at C-terminus to exclude the S1/S2 cleavage site. The Alpha S1 construct 2 (SEQ ID NO. 4) contained a unique signal sequence based on that of interleukin-2 (IL-2) in place of amino acids 1-13 of Alpha S1. Nucleotide sequences were optimized for HEK293 cell expression to enhance post translation modifications of the S1 proteins to enhance signal peptide cleavage, glycosylation, and disulfide bond formation.

Total expression of Alpha S1 was better using Alpha S1 construct 1 (SEQ ID NO. 3) than it was using with Alpha S1 construct 2 (SEQ ID NO. 4). The total yields of unpurified Alpha S1 produced using construct 1 and construct 2 were from about 90-120 mg/L and from about 50-60 mg/L, respectively. Overall purity was higher with Alpha S1 construct 1, which was used to produce higher levels of monomeric Alpha S1 than could be achieved using Alpha S1 construct 2.

Higher levels of soluble aggregates and S1 oligomers were also produced using the Alpha S1 construct 2, which reduced the overall final yield and would not be ideal for manufacturing processes. Therefore, it was concluded that the best design was to keep the canonical leader sequence for optimal post-translational modification processing resulting in higher monomeric species in the purified product and increased overall yield.

The Delta S1 construct (SEQ ID NO. 5) included the canonical leader sequence and produced expression levels of 90-120 mg/L, similar to Alpha S1 construct 1. In addition, the Delta S1 construct did not have high levels of aggregated S1 or multimeric species.

Unless otherwise specified, the data below were generated using the Alpha S1 construct 1 corresponding to SEQ ID NO. 3.

### Example 3: Expression of ACE2 and SARS-CoV-2-Spike 1 (S1) in Mammalian Cells

Vectors and expression systems were as described in Examples 1 and 2. Serum-free suspensions of HEK cells were transfected with a cationic lipid-based reagent and incubated at 37° C. Approximately 5-7 days after transfection, clarified supernatant was harvested for purification. Supernatant was clarified at 4,000 rpm for 30 minutes and then filtered using a bottle filter with 0.22μ or 0.45μ PFTE membrane.

### Example 4: Purification of Recombinant Human ACE2s and SARS-CoV-2-Spike 1 (S1) Proteins Using Affinity Chromatography

Nickel Sepharose® resin was used to purify the ACE2s and Spike 1 recombinant proteins. The resin was equilibrated with 20 mM Tris buffer (pH 8.0/150 mM NaCl). Clarified culture supernatant was loaded and column was washed with two steps prior to a step gradient with various concentrations of imidazole. Pooled fractions containing the largest amount of target were further purified in a second chromatography step (polish chromatography) wherein Superdex® S200 was used to isolate monomeric ACE2 and Spike 1 proteins. Pooled fractions containing monomer were concentrated and characterized by size exclusion (SEC)-HPLC and SEC-MALS. Purity was determined to be >99.0% monomeric ACE2 and >89.0% monomeric Spike-1.

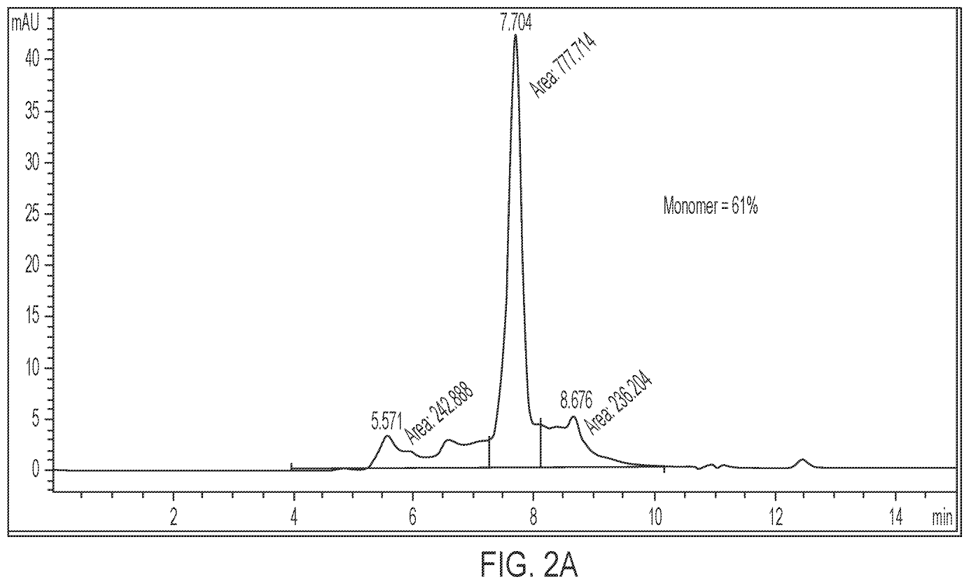

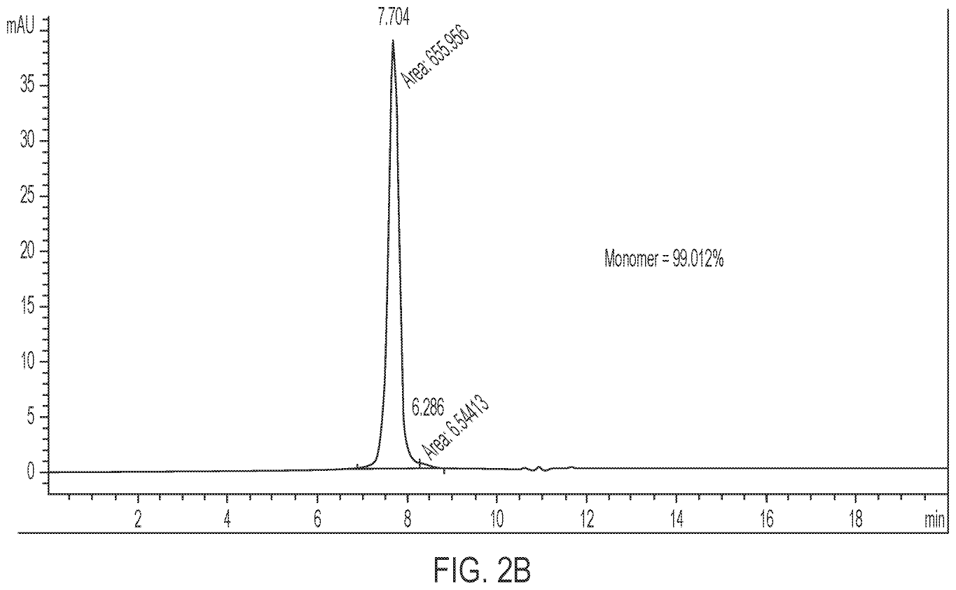

FIGS. 2A and 2B depict chromatographs of purified recombinant ACE2 protein (ACE-GS-8×HN; SEQ ID NO. 1) before and after polish chromatography. Before polish chromatography, samples contained aggregates, dimers and monomers (FIG. 2A). After polish chromatography, samples contained almost exclusively monomeric ACE2 (FIG. 2B).

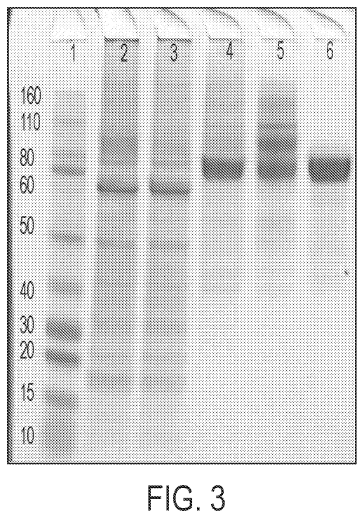

FIG. 3 depicts SDS-PAGE of purified recombinant ACE2 protein (hACE-GS-6×HIS; SEQ ID NO. 2). Lane 1: protein standard; Lane 2: Sample load; Lane 3: Sample column flow through, nickel resin; Lane 4: hACE-GS-6×HIS elution, nickel resin; Lane 5: hACE-GS-6×HIS SEC polish, monomer and dimer, non-reduced; Lane 6: hACE-GS-6×HIS SEC polish, monomer, non-reduced.

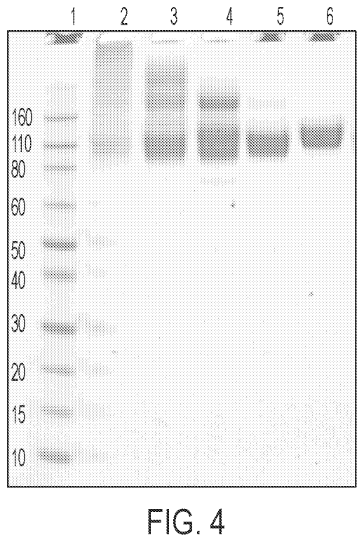

FIG. 4 depicts SDS-PAGE of purified recombinant SARS-CoV-2 S1 construct 1 (SEQ ID NO. 3). Lane 1: protein standard; Lane 2: S1-HIS, aggregated, non-reduced; Lane 3: S1-HIS, monomer, dimer, trimer, non-reduced; Lane 4: S1-HIS, monomer, dimer, non-reduced; Lane 5: S1-HIS SEC, monomer, non-reduced; Lane 6: S1-HIS SEC, monomer, reduced.

### Example 3: Biotinylation of ACE2

Purified recombinant human ACE2 was biotinylated for 20-40 minutes at room temperature using 10-fold excess pre-activated biotin. To remove excess biotin, the complex was applied to a gel filtration desalting column, where the biotinylated ACE2 eluted prior to the free biotin. Biotin incorporation was determined to be in the range of 1-10 biotins per ACE2 [mole:mole].

### Example 4: Development of Assay Conditions Using Purified Recombinant Proteins and Purified Anti-Spike Monoclonal Antibodies

### 4.1 Preparation of S1-Coated Plates

Using a 96-well high-binding plate, 3 concentrations of recombinant S1 were coated overnight at room temperature in PBS, pH 7.4, 100 μL per well. The following day the wells were blocked with standard blocking buffer for 2-24 hours, 300 μL per well. Prior to adding ACE2-biotin, blocking buffer was aspirated or washed 3×250 μL of standard ELISA wash buffer containing detergent and air dried for 24 hours or used immediately.

### 4.2 Determination of Optimal ACE2-Biotin Concentration

EC₂₅, EC₅₀ and EC₇₅ concentrations were determined by serial dilution of ACE-biotin. ACE-biotin was diluted in standard assay buffer diluent. Serial dilutions of ACE-biotin were performed in a non-binding 96-well plate. 50 μL per well of each dilution were transferred to the Spike 1 coated wells in duplicate, with a 2-fold series of titrations in the range of 7.5 nM to 0.06 nM. EC₅₀ was determined to be 0.5 nM of ACE-biotin. After 1 hour at room temperature the plate was washed using a plate washer and standard ELISA assay wash buffer. Detection of the bound ACE-biotin was by addition of horseradish peroxidase conjugated to streptavidin (SA-HRP) in 4 conditions, for 30 minutes at room temperature, removed from light. Negative wells did not contain ACE-biotin. The plates were washed extensively after incubation. Chromogenic amplification was performed using standard 3,3′,5,5′-tetramethylbenzidine (TMB). Development was stopped after 30 minutes using 2N sulfuric acid. OD values were read using a standard plate reader at wavelength 450 nm. The optimal condition for SA-HRP was determined to be 1:10,000 in 100 μL per well.

### 4.3 Reagent and Assay Condition Optimization Using Purified Anti-Spike 1 Monoclonal Antibodies

Eight purified anti-S1 monoclonal antibodies (mAbs) were screened for ACE-biotin inhibition on the Spike 1 coated plates. Duplicate 2, 3 or 4-fold serial dilutions of antibodies were prepared in a non-binding 96-well plates using standard ELISA sample diluent buffer. 50 uL of each dilution was transferred to the Spike 1 coated plate and incubated for 30 minutes at 37° C. to enhance rapid binding of the anti-Spike antibodies. The plate was removed to room temperature where ACE-biotin was added in equal volume to the anti-Spike 1 mAbs and final concentration of 0.5 nM for 1 hour. The plate was washed, and detection of bound ACE-biotin was performed using SA-HRP as described in Example 4.2. No neutralization of activity was determined in wells that no ACE-biotin was applied. Non-specific signal due to detection reagent was determined in wells that contained Spike 1 protein and SA-HRP without the addition of anti-Spike mAbs or ACE-biotin. A range of neutralization activity was demonstrated by the titration of mAb concentration. Antibodies that bound to RBD domain in SARS-CoV-2 Spike protein were identified by the titration of the mAb concentration versus a constant amount of ACE-biotin. This was demonstrated by the reduction of chromogenic substrate in correlation with mAb concentration thereby indicating that ACE-biotin was being blocked from binding RBD in Spike 1. Those antibodies that did not bind to the RBD domain did not provide any blocking of ACE-biotin binding to Spike 1 and therefore a chromogenic color change was observed with the addition of SA-HRP and TMB. One monoclonal antibody (Rabbit-anti-S1 clone 4B2) was selected for consecutive plate controls conferring neutralizing activity by the inhibition of ACE-biotin binding to Spike 1 in the range of 0.1 nM to 16.7 nM.

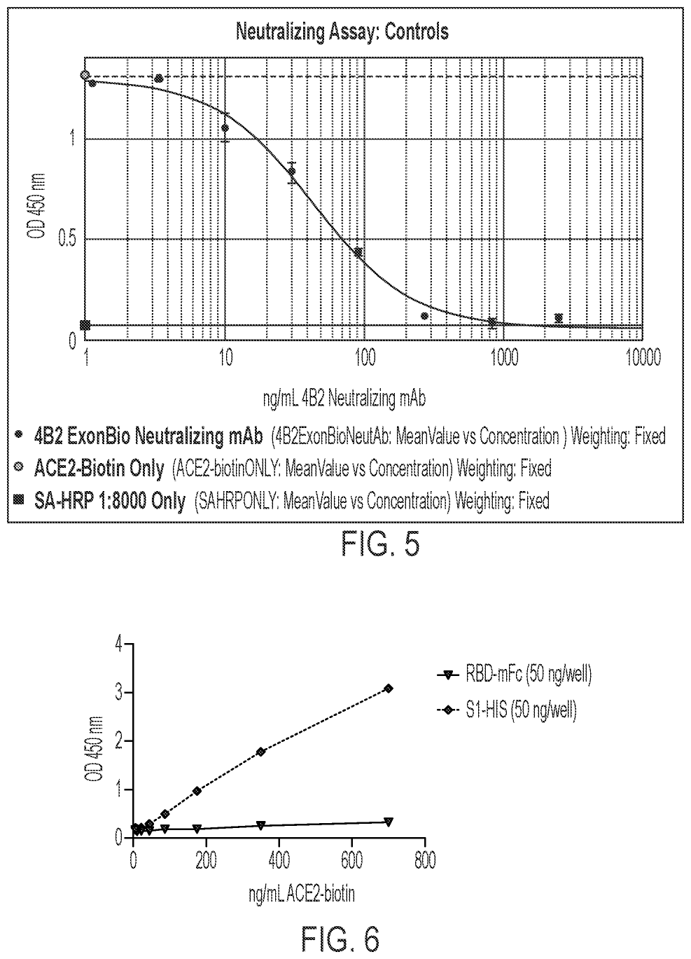

FIG. 5 depicts the EC₅₀ curve using positive control neutralizing antibody. Recombinant S1-HIS was coated on a 96-well plate at 200 ng/well. ACE2-biotin was pre-incubated with Rabbit-anti-S1 clone 4B2 at different concentrations via serial dilutions. Optical density (OD) values are plotted as dose-response curve. In the negative control, the ACE2-biotin binds fully to S1 on the plate and produces an OD value of ˜1.5 (grey circle; upper line). Control wells without ACE2-biotin or anti-S1 antibody had OD values are <0.100 (square; lower line).

As depicted in FIG. 6, recombinant ACE2-biotin demonstrates superior binding to recombinant SARS-CoV-2 S1 compared to commercially available SARS-CoV-2 RBD-mFc only. Commercially available RBD protein was sourced from Genscript and claims to bind ACE2 at concentrations as low as 1 ng/mL. This claim was not repeatable as shown above, which has >40-fold more RBD-mFc added per well. This data indicates that recombinant S1 protein, which includes secondary structure around the RBD, is the superior format for a surrogate neutralization assay. Protein folding using S1 may be superior due to the associated surrounding amino acids supporting the proper folding of the RBD domain.

### Example 5: Neutralization Activity Using Human Serum

### 5.1 Serum Sample Collection

Donors were screened for previous COVID-19 infection, COVID-19 vaccination manufacturer, and date of last COVID-19 vaccine dose. Blood was collected from donors under IRB protocol into blood collection tubes. Serum was separated from plasma after 30 minutes at room temperature by centrifugation at 1,000×g for 10 minutes. Serum was transferred to small polypropylene tubes for storage at −80 C.

5.2 iNab Assay

Dilutions of control neutralizing anti-S1 monoclonal antibody (mAb) and donor serum were prepared using ELISA sample diluent solution. 50 μL per diluent in duplicate were transferred to wells of Spike-1 coated 96-well plates. Reactions were incubated at 37° for 30 minutes. ACE-biotin was added to the reactions at 25% inhibitory concentration (IC₂₅) in equal volume to the serum. Negative controls did not receive any serum or ACE-biotin in the wells. Positive controls included ACE-biotin but no control neutralizing mAbs in the wells. Reactions were incubated for 1 hour at room temperature. The plate was washed and 100 μL SA-HRP (1:10,000 dilution) was added to reactions. The reactions were incubated at room temperature for 30 minutes away from the light. 100 μL TMB was added to the reactions to induce the colorimetric change, and the reactions were incubated at room temperature for 30 minutes away from the light. Reactions were quenched with 100 μL 2N sulfuric acid. Within 5 minutes of quenching the reactions, the absorbance at 450-630 nm was measured.

Percent inhibition was determined by

$$\% Inhibition = ( 1 - \frac{S - {C}_{min}}{{C}_{max} - {C}_{min}} ) \times 100 ;$$

where S is the experimental signal, and Cₘᵢₙ and Cₘₐₓ are the negative and positive controls, respectively.

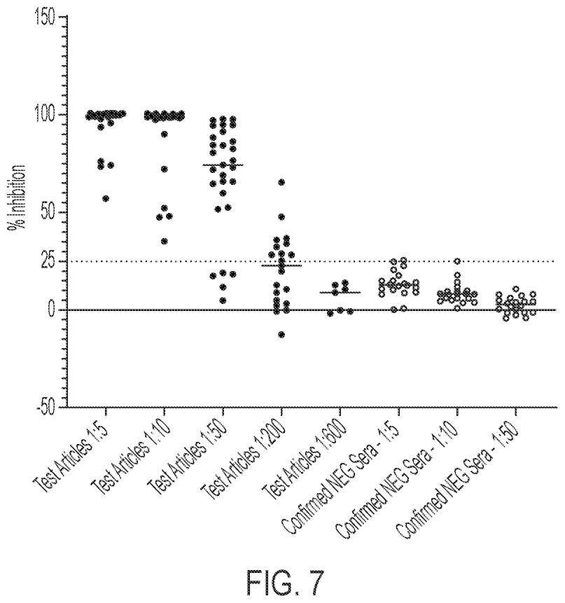

FIG. 7 depicts results of serum tested using iNab Assay. Test articles collected under IRB approved protocol from persons who had either recovered from COVID-19 or had received full vaccination regime of 1 dose (J&J, Moderna) or 2 doses (Pfizer) or a combination of naturally infected/recovered plus vaccinated. Confirmed negative serum samples collected under IRB approved protocol from persons who were COVID-19 negative and not vaccinated. Titration of serum showed decreased titer of neutralizing antibodies as the dilution increases. Dilutions of [1:50] and larger showed false negatives as the % neutralizing activity drops under 25% cut-off due to titer levels. Dilution [1:10] was ideal for use in iNab because it showed differentiation among high, medium, and low anti-Spike 1 titers as well as it resulted in 100% correlation with confirmed negative samples falling under the 25% cut-off for neutralization.

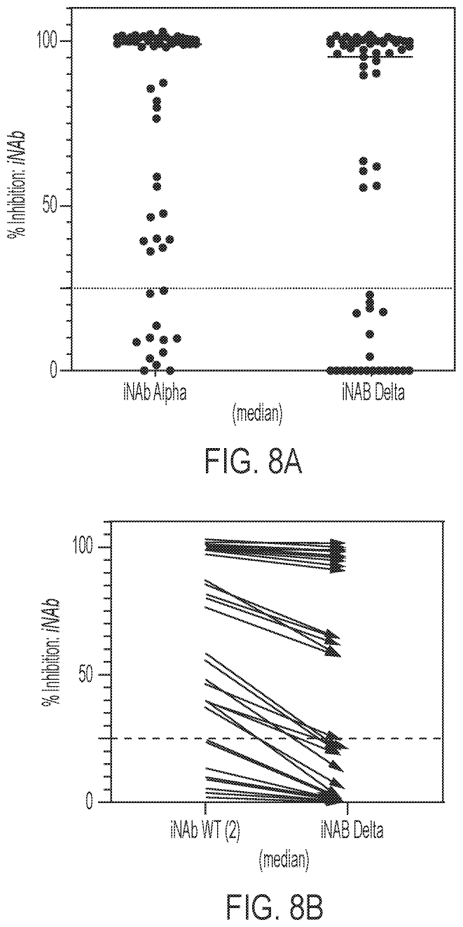

FIG. 8A shows dot plot comparison of serum samples tested in iNab at dilution [1:10] using S1 Alpha or S1 Delta for ACE2 binding. All samples below 25% cut-off were confirmed negative for SARS-CoV-2 Alpha anti-Spike antibodies. Not all samples below 25% cut-off were confirmed negative for SARS-CoV-2 Delta anti-spike antibodies. The iNab assay provided evidence of diminished neutralization response for some donors against Delta variant. These are reflected as samples that directionally fall under the cut-off line established in the iNab assay. Because of the unique format and reagent design, the sensitivity of iNab, there are less false negatives compared to other developed assays which have a cut-off at 30% (Genscript C-Pass).

FIG. 8B shows the Before-and-After Graph representation of Fig A. Grouped lines shows three distinct populations of anti-spike titers representing high, medium, and low responders to COVID-19, naturally infected, vaccinated, or both. Arrow directions show that neutralizing activity generally decreases when exposed to the S1 Delta protein vs S1 Alpha, as expected. This data provides evidence that the recombinantly expressed S1 protein in Delta variant form works in surrogate neutralization assay similarly to the Wuhan S1. In addition, there is no need to have RBD domain exclusively in order to evaluate new variants for neutralizing activity, which is the format of many published neutralization assays for SARS-CoV-2.

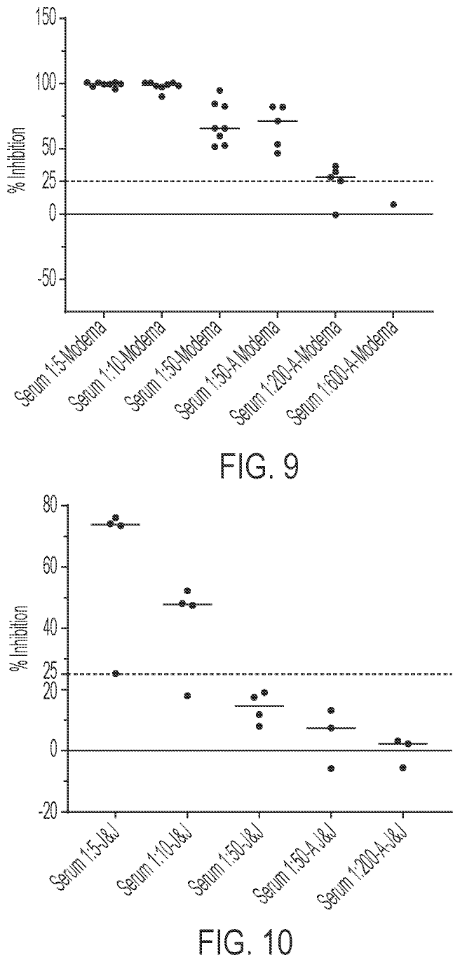

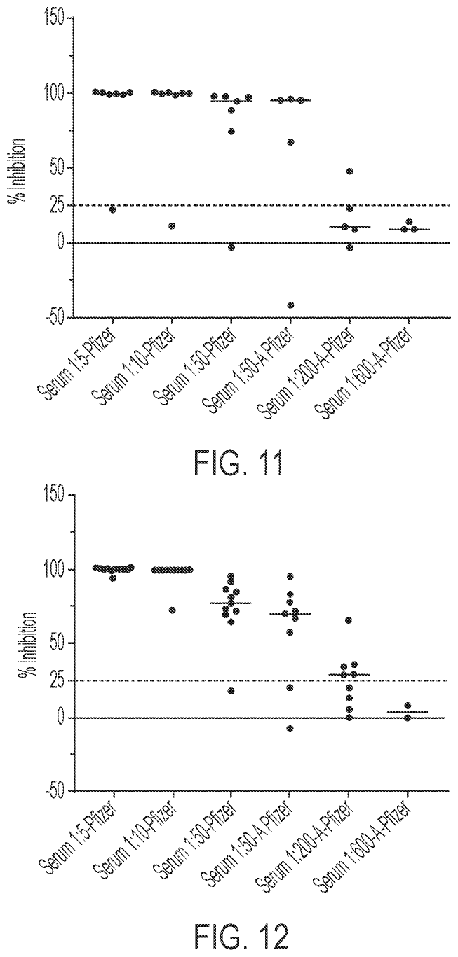

Dot plots shown in FIGS. 9-12 represent iNab results using serum samples from vaccinated donors. Samples are divided by vaccine type and listed by manufacturer. Several dilutions of serum were tested to confirm the optimal test condition was [1:10] dilution of serum in iNab. These samples were also tested using live virus and showed 100% correlation between iNab and Vero E6 VNT assay as described below FIG. 14.

### 5.3 Validation Using Virus Neutralization Test (VNT) Assay

VNT assay was performed in a BSL3 laboratory using SARS-CoV-2 isolate New York-PV08449/2020 obtained from BEI Resources. 70-90% confluent Vero E6 cells were grown in 96-well flat bottom tissue culture treated plates. Cells were seeded at 1×10⁴ cells/well in DMEM or F12 medium, grown overnight at 37° C., 5% CO₂. Heat inactivated donor serum was diluted in non-binding 96-well plates using DMEM or F12 medium in 2-fold serial dilutions in range of [1:10] to [1:10,240]. 50 μL to 100 uL of diluted sera was transferred to a new 96-well plate in quadruplicate for each dilution. Virus at target concentration was added to the diluted sera in equal volume amounts and incubated at 37° C. for up to one hour to allow SARS-CoV-2 RBD to bind to neutralizing serum antibodies. The old media on the Vero E6 plates was discarded. After the one-hour pre-incubation of sera with virus, 100 uL of pool was transferred to Vero E6 plates. Negative control wells did not have any virus added. Plates were incubated at 37° C., 5% CO₂ for 5-7 days before reading for serum neutralization and cytotoxicity effects. In addition, back-titer plates were used to determine actual viral titer used in the assay vs the target. Viral titer was calculated using Excel based Reed-Muench-Lindenbach Version 2008 for TCID₅₀. Neutralizing titer was calculated using TCID₅₀ using the Spearman-Karber method and recorded as MN50/mL to represent the neutralizing dose per titer of the serum at >=50% against the virus.

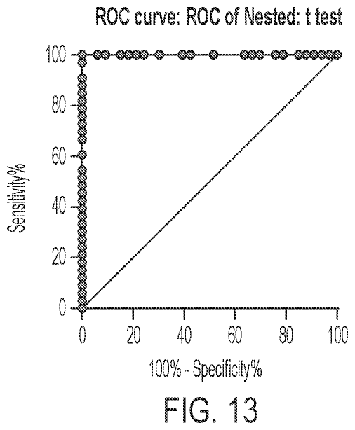

FIG. 13 shows the ROC curve for representative COVID-19 vaccinated serum tested by iNab assay as they correlate to gold standard Vero E6 VNT assay. ROC data is reflected in Table 1. 33/33 vaccinated individuals had neutralizing responses of greater than 25% by iNab and had Wuhan or Delta MN 50/mL titers as determined by the gold standard BSL3 based VNT assay. The same correlation was produced among the non-vaccinated/non-infected donors. This data supports the use of high-throughput plate-based assays in BSL2 labs to screen for neutralizing antibodies. iNab results are conclusive in <4 hours compared to the gold standard VNT BSL3 cell culture-based assay which takes several days. For immunocompromised patients that may be time constrained medical procedures, the iNab would provide a quick and easy substitution for the lengthy alternative.

TABLE 1

| Area under ROC Curve (infected/vaccinated) | Value |
|---|---|
| Area | 1.000 |
| Std. Error | 0.000 |
| 95% confidence interval | 1.000 to 1.000 |
| P value | <0.0001 |
| Controls (MN₅₀/mL) | 33 |
| Patients (% inhibition serum) | 33 |
| Missing Controls | 0 |

1-27. (canceled)

28. An assay method for assessing the strength of an immune response to SARS-CoV-2 virus in a subject, which has been exposed, or potentially exposed, to SARS-CoV-2 virus, the method comprising:
a) contacting a solid substrate, on which a plurality of SARS-CoV-2-Spike 1 (S1) subunit proteins (S1 antigens) are bound to the surface of the substrate, with a biological sample from the subject, under reaction conditions to allow S1 antigen-specific antibodies, if present in the biological sample, to bind the S1 antigen, bound to the substrate, to form S1 antigen-antibody complexes on the surface of the substrate;
b) contacting the S1 antigen-antibody complexes on the surface of the substrate with a solution comprising a plurality of biotinylated angiotensin-converting enzyme 2 (ACE2) proteins under conditions to allow the biotinylated ACE2 proteins to compete for binding to the S1 antigen with the complexed S1 antigen-specific antibodies in (a);
c) contacting the solution of (b) with a solution comprising streptavidin labeled with a detection moiety, under conditions to allow labelled streptavidin-biotin complexes to form; and
d) detecting the labelled streptavidin-biotin complexes, wherein the strength of the immune response to S1 antigen corresponds to the level of label detected, wherein the level of detected label corresponds to inhibition of ACE2 protein binding to the S1 antigen by the S1 antigen-specific antibodies in the biological sample.

29. The assay method of claim 28, wherein the biological sample is blood or serum.

30. The assay method of claim 28, wherein the biological sample is diluted from 1:10 to 1:20,000.

31. The assay method of claim 28, wherein the S1 antigens are S1 subunit monomers.

32. The assay method of claim 28, wherein the S1 antigens comprise one or more S1 subunit proteins of any one of the following SARS-CoV-2 variants: WHO Alpha (WT); WHO Delta; U.K. (alpha, B.1.1.7); South African (beta, B.1.351); Brazil (gamma, P.1); India (delta, B.1.617.2); California (epsilon, B.1.429/427); and Epsilon; Epsilon BA.1; Epsilon BA.2; Epsilon BA.3; Epsilon BA.4; and Epsilon BA.5.

33. The assay method of claim 31, wherein the amino acid sequences of the S1 subunit monomers have at least 80%, at least 85%, at least 90%, at least 95%, at least 96%, at least 97%, at least 98%, or at least 99% sequence identity to the amino acid sequence of any one of SEQ ID Nos: 3-5.

34. The assay method of claim 28, wherein the S1 antigens comprise an N-terminal affinity tag or a C-terminal affinity tag.

35. The assay method of claim 28, wherein the S1 antigens comprise S1 subunit receptor-binding domains (RBD).

36. The assay method of claim 28, wherein the solid substrate is glass or a polymer.

37. The assay method of claim 36, wherein the solid substrate is a polymer selected from cellulose, polyacrylamide, nylon, polystyrene, polyvinylchloride, and polypropylene.

38. The assay method of claim 28, wherein the solid support is the surface of a tube, a bead, a disc, or a microplate.

39. The assay method of claim 28, wherein the ACE2 protein amino acid sequences of the biotinylated ACE2 proteins have at least 80%, at least 85%, at least 90%, at least 95%, at least 96%, at least 97%, at least 98%, or at least 99% sequence identity to the amino acid sequence of any one of SEQ ID Nos: 1 or 2.

40. The assay method of claim 39, wherein the biotinylated ACE2 proteins comprise an N-terminal affinity tag or a C-terminal affinity tag.

41. The assay method of claim 28, wherein the streptavidin labeled with a detection moiety is streptavidin-HRP.

42. The assay method of claim 28, further comprising performing the assay method with a negative control and a positive control, wherein the negative control does not comprise biotinylated ACE2 proteins, and the positive control comprises known neutralizing antibodies that are specific for the RBD of the SAR-CoV-2 S1 subunit.

43. The assay method of claim 42, wherein percent inhibition of antibodies in the biological sample is calculated using the following formula:
$$\% Inhibition = ( 1 - \frac{S - {C}_{min}}{{C}_{max} - {C}_{min}} ) \times 100 ;$$
wherein S is the signal detected using the biological sample, and Cₘᵢₙ and Cₘₐₓ are the negative and positive controls, respectively.

44. The assay method of claim 43, wherein if the strength of the immune response to SARS-CoV-2 virus in a subject is low, the subject is administered treatment for SARS-CoV-2, wherein a low strength immune response to SARS-CoV-2 virus is 25% or less inhibition.

45. The assay method of the method of claim 43, wherein the subject is need of an organ transplant, and wherein,
if the strength of the immune response to SARS-CoV-2 virus in the subject is high, then the subject receives the organ transplant, or
if the strength of the immune response to SARS-CoV-2 virus in a subject is low, then the subject does not receive the organ transplant;
wherein a high strength immune response to SARS-CoV-2 virus is 75% or more, 80% or more, 85% or more, 90% or more, 95% or more, or 100% inhibition, and
a low strength immune response to SARS-CoV-2 virus is 25% or less inhibition.

46. A kit for assessing the strength of an immune response to SARS-CoV-2 virus in a subject, the kit comprising:
(A) a plurality of recombinantly-produced SARS-CoV-2 Spike 1 (S1) subunit monomers bound to a solid substrate;
(B) a plurality of biotinylated angiotensin-converting enzyme 2 (ACE2) proteins; and
(C) a solution comprising streptavidin labeled with a detectable moiety.


SEQUENCE LISTING (5 sequences)

SEQ ID NO: 1 - protein, 625 aa, synthetic construct
MYRMQLLSCIALSLALVTNSSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDGSHNHNHNHNHNHN

SEQ ID NO: 2 - protein, 629 aa, synthetic construct
MYRMQLLSCIALSLALVTNSSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGXLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDGGGGSGGGGSHHHHHHH

SEQ ID NO: 3 - protein, 680 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYHHHHHH

SEQ ID NO: 4 - protein, 688 aa, synthetic construct
MYRMQLLSCIALSLALVTNSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYHHHHHH

SEQ ID NO: 5 - protein, 678 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYHHHHHH